(19) United States
(12) Patent Application Publication
(10) Pub. No.: US 2013/0165776 A1
(43) Pub. Date: Jun. 27, 2013

(54) CONTRACTION STATUS ASSESSMENT

(71) Applicant: Andreas Blomqvist, Taby (SE)

(72) Inventor: Andreas Blomqvist, Taby (SE)

(21) Appl. No.: 13/334,654

(22) Filed: Dec. 22, 2011

(51) Int. Cl. A61B 5/02; A61B 5/0428; A61B 8/02

(52) U.S. Cl. 600/437; 600/508; 600/509

(57) ABSTRACT

An implantable medical device receives at least one sensor signal representing inter-movement between a basal region of a heart ventricle and a ventricle apex during at least a portion of a systolic phase of a cardiac cycle. A parameter processor calculates a contraction status parameter value based on the at least one sensor signal. This contraction status parameter value represents an elongation of the ventricle following onset of ventricular activation during a cardiac cycle. The contraction status parameter value is stored in a memory as a diagnostic parameter representing a current contraction status of a subject's heart.

### BACKGROUND OF THE INVENTION

1. Field of the Invention

The present embodiments generally relate to cardiac monitoring and in particular to the assessment of contraction status of a subject's heart.

2. Description of the Prior Art

Approximately 14 million people in Europe suffer from heart failure (HF). More than 3 million new cases are diagnosed every year. The 5-year mortality of HF is approximately 50%. In screening for HF patients the echocardiography-based measure ejection-fraction (EF) is the gold standard for evaluation of systolic function. The traditional HF symptoms or, more precisely, systolic HF symptoms are fatigue, shortness of breath, excessive fluid retention, among others, and an EF below 30 or 35%.

These patients have typically disturbances in their contraction patterns or reduced systolic function. It has also lately been hypothesized that in fact pacemakers operating in the DDD pacing mode (dual pace, dual sense and dual inhibit) and with right ventricular (RV) pacing or RV and right atrial (RA) pacing may in fact induce heart failure by disturbing primarily the systolic function and impairing contraction.

The introduction of cardiac resynchronization therapy (CRT) devices has served as a compliment to drug therapy for HF patients. Conventional biventricular CRT involves pacing from the RV apex, the transvenous left ventricle (lateral or postero-lateral vein), and the right atrium, and is directed towards resynchronization of the right and left ventricles by optimizing systolic contraction.

In spite of CRT becoming a more widely accepted standard of care, it is still both costly and time consuming so it is very unlikely that CRT devices in the near future will be implanted instead of DDD-pacemakers to avoid the aforementioned potential risk of developing heart failure.

There is, thus, a need for a technique that can be used to monitor the contraction status of a subject in order to detect cardiac conditions that cause a reduction in systolic function and contraction, including the above-mentioned heart failure conditions and possible pacemaker-induced conditions.

Emilsson et al., “Mitral annulus motion versus long-axis fractional shortening”, Exp Clin Cardiol, Vol. 11, No. 4, 302-304, 2006 discloses that the long-axis fractional shortening (FS_{L}) of the left ventricle can be used to assess left ventricular systolic function and shows correlation with EF. The parameter FS_{L} represents the ratio between the echocardiograph recording of mitral annulus motion (MAM) and the end-diastolic length of the ventricle. MAM in turns represents the left atrioventricular plane displacement.

U.S. Pat. No. 7,445,605 relates to detecting and monitoring cardiac dysfunction using motion sensors recording signals representative of the movement of the apex of the heart. The document discloses that the shortening of the heart during contraction and the particular movement of the apex of the heart during contraction can be used to detect various cardiac dysfunctions including ischemia and congestive heart failure.

There is, though, still a need for a technique that can be used to assess the contraction status of a subject's heart.

### SUMMARY OF THE INVENTION

It is a general objective to assess the contraction status of a subject's heart.

This and other objectives are met by embodiments disclosed herein.

An aspect of the embodiments relates to an implantable medical device (IMD) comprising a sensor connector connectable to a sensor arrangement comprising at least a first sensor unit. The sensor arrangement is configured to output at least one sensor signal representing inter-movement between a basal region of a heart ventricle and an apex of the ventricle during at least a portion of a systolic phase of a cardiac cycle. The IMD also comprises a parameter processor configured to calculate a contraction status parameter value based on the at least one sensor signal. The contraction status parameter value represents an elongation of the ventricle following onset of ventricular activation during the cardiac cycle. This contraction status parameter value is stored in a memory as a diagnostic parameter representing a current contraction status of the heart.

Another aspect of the embodiments defines a method of assessing contraction status of a subject's heart. The method comprises determining a distance signal representing a distance between an apex of a heart ventricle and a basal region of the ventricle during at least a portion of a systolic phase of a cardiac cycle. A contraction status parameter value is calculated based on the contraction status parameter value. The contraction status parameter value represents an elongation of the ventricle following onset of ventricular activation during the cardiac cycle and is used to assess the contraction status of the subject's heart.

The embodiments provide early and sensitive means of detecting systolic dysfunction that affects cardiac contraction.

### BRIEF DESCRIPTION OF THE DRAWINGS

FIG. 1 is a schematic overview of a subject and an implantable medical device according to an embodiment.

FIG. 2 is a schematic block diagram of an implantable medical device according to an embodiment.

FIG. 3 schematically illustrates an embodiment of calculating a distance signal representing vertical displacement between right atrium and right ventricle.

FIGS. 4A and 4B illustrate a distance signal plotted over time for a healthy subject (FIG. 4A) and a subject having impaired cardiac contraction (FIG. 4B).

FIG. 5 is a flow diagram illustrating a method of assessing contraction status according to an embodiment.

FIG. 6 is a schematic overview of a subject and a contraction status assessing system according to another embodiment.

### DESCRIPTION OF THE PREFERRED EMBODIMENTS

Throughout the drawings, the same reference numbers are used for similar or corresponding elements.

The present embodiments generally relate to cardiac monitoring and in particular to the assessment of contraction status of a subject's heart. This contraction status assessment is performed by monitoring the inter-movement of different parts of a ventricle of a subject's heart during at least a portion of a cardiac cycle. Thus, how these different parts move relative each other can be used to assess the contraction status of the subject's heart.

Different models exist to describe how the heart moves during a cardiac cycle. One of these models, denoted the Torrent-Guasp modes, states that a healthy cardiac contraction starts with an initial torsion motion. This motion originates from the base of the heart, also identified as the basal region of a ventricle or the valve plane, and propagates in a helical manner down and around the heart towards the apex. This torsion motion is followed by a contraction from the apex and upwards along the long axis of the heart.

The initial torsion motion is an important feature that is part of the systolic function. This torsion motion becomes impaired or disturbed in presence of various cardiac conditions, such as left bundle branch block (LBBB), severe ischemia or RV-pacing-induced cardiac disturbances for pacemakers operating in the DDD mode.

The present embodiments monitor this initial torsion motion by determining a diagnostic parameter that reflects how this initial torsion motion is propagating during a cardiac cycle. In more detail, in a healthy heart the initial torsion motion causes an initial elongation of the ventricles by providing a relative elongating motion between the basal region of a ventricle (valve plane) and the apex of the ventricle. This comparatively short and initial elongation is then accompanied with a significant shortening of the ventricles along the long axis of the heart during the subsequent part of the contraction when the ventricles are contracting from the apex up towards the valve plane.

Experimental data indicates that the initial elongation of the ventricles is mainly due to a deflection of the basal region of the ventricles, i.e. the valve plane of the heart. Thus, this basal ventricle region is moved upwards towards the atria of the heart, whereas the apex of the ventricles is mainly stationary during this initial part of the systolic phase of a cardiac cycle. The upward movement of the basal region together with the substantially stationary apex implies that the ventricles will lengthen and elongate prior to the following contraction where the basal region and the apex are moved towards each other.

Thus, a diagnostic parameter that represents this elongation can be used to represent and monitor a current contraction status of the heart. Hence, any disturbances or attenuations in the initial elongation as detected based on the diagnostic parameter can be a marker for deleterious cardiac conditions, which may eventually lead to heart failure.

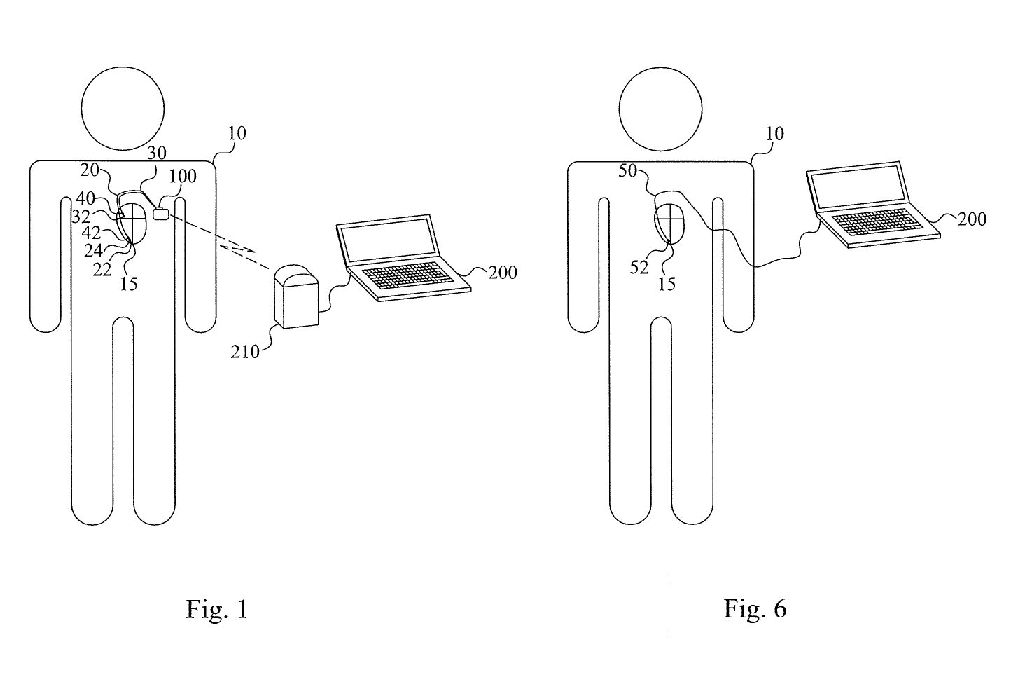

FIG. 1 is a schematic overview of a subject, represented by a human subject 10 having an implantable medical device (IMD) 100 according to the embodiments. The IMD 100 is implanted in the subject 10 in order to provide pacing therapy to the subject's heart 15. The IMD 100 can be in the form of a pacemaker or an implantable cardioverter-defibrillator (ICD). The IMD 100 is, during operation in the subject's body, connected to an implantable medical lead or cardiac lead 20, 30 having at least one pacing electrode 22, 24, 32 arranged in or in connection with the subject's heart 15 to deliver pacing pulses to the heart 15 and/or sense electric activity of the heart.

In FIG. 1, the IMD 100 has been exemplified as being connectable to a right ventricular (RV) lead 20 and a right atrial (RA) lead 30. An RV lead 20 is typically provided inside the right ventricle of the heart 15 and comprises one or more electrodes 22, 24 that can be used by the IMD 100 to apply pacing pulses to the right ventricle and/or sense electrical activity from the right ventricle. An RA lead 30 having at least one electrode 32 arranged in or in connection with the right atrium, can be used by the IMD 100 in order to provide atrial pacing and/or sensing. Instead of or as a complement to an RA lead, the IMD 100 can be connected to a left atrial (LA) lead. Furthermore, instead of or as a complement to the RV lead 20 the IMD 100 could be connected to a left ventricular (LV) lead. Such a LV lead is generally provided on the outside of the heart 15 typically in the coronary venous system, e.g. in a left lateral vein or a postero-lateral vein. The LV lead enables the IMD 100 to apply pacing pulses to the left ventricle and sense electrical activity from the left ventricle.

The particular implantable medical lead(s) which are connectable to the IMD 100 are not decisive for the present embodiments. Thus, the IMD 100 could be, in operation in the subject body, connected to a single implantable medical lead 20, 30 or multiple, i.e. at least two, implantable medical leads 20, 30. In fact, the IMD 100 does actually not need to be connected to any implantable medical lead at all if it is merely employed for diagnostic purposes by monitoring the contraction status of the heart 15.

The IMD 100 is connectable to a sensor arrangement comprising at least a first sensor unit 40 but preferably comprising the first sensor unit 40 and a second sensor unit 42. The first sensor unit 40 is then arranged in connection with the basal region of the ventricles, i.e. preferably in connection with a portion of the valve plane separating the ventricles from the atria.

In the case of a sensor arrangement with multiple sensor units 40, 42, these are then configured to be implanted at different sites in or in connection with the subject's heart 15 to measure the distance between an apex of a ventricle and a basal region of the ventricle during at least a portion of a systolic phase of a cardiac cycle. The sensor arrangement will be described further herein.

FIG. 1 additionally illustrates a non-implantable data processing device 200, such as in the form of a programmer, a home monitoring device or a physician's workstation. The data processing device 200 comprises or is connected to a communication module or device 210 that is capable of wirelessly communicating with the IMD 100, preferably through radio frequency (RF) based communication or inductive telemetry. The data processing device 200 can then use the communication module 210 in order to interrogate the IMD 100 for diagnostic data recorded by the IMD 100 employing the sensor arrangement and/or any electrodes 22, 24, 32 of the connected implantable medical lead(s) 20, 30. Furthermore, the data processing device 200 can be used to program the IMD 100, such as by setting one or more programmable operating parameters. According to the present embodiments, the IMD 100 can in particular transmit information of the contraction status of the heart 15 to the data processing device 200 for processing therein, such as display to the subject's physician.

The communication module 210 and the data processing device 200 can be separate devices as illustrated in FIG. 1, either wired connected or using a wireless connection, such as Bluetooth®, an infrared (IR) connection or an RF connection. In an alternative embodiment, the functionality and equipment of the communication module 210 can be housed within the data processing device 200.

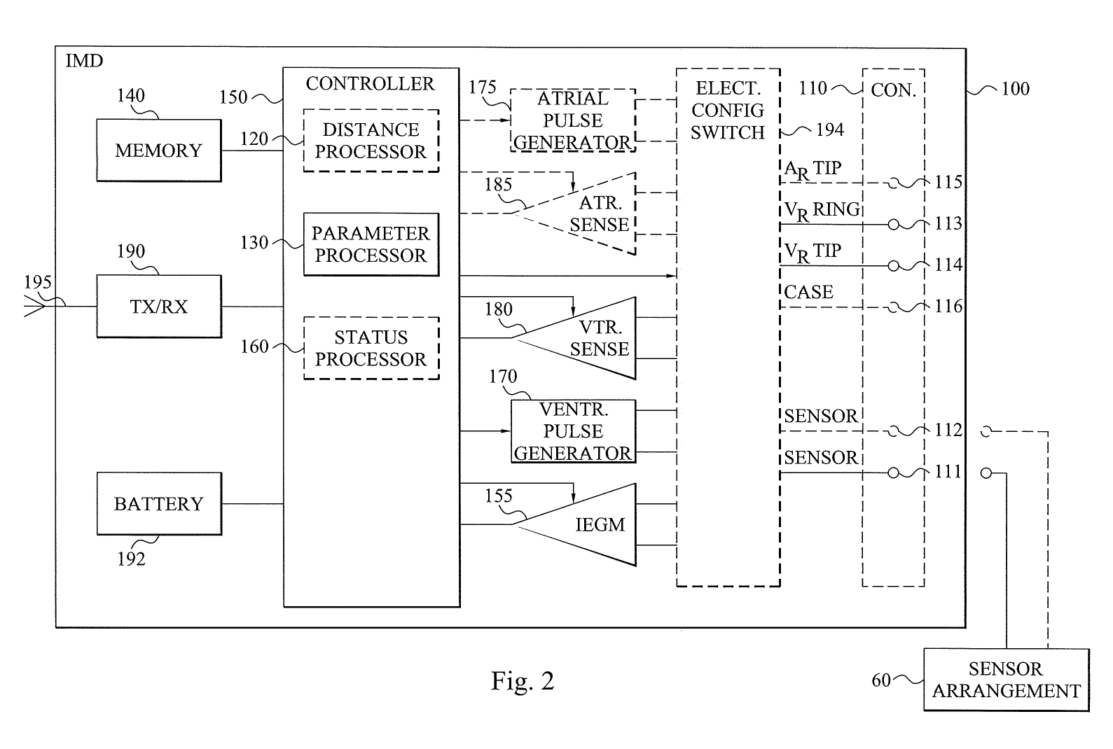

FIG. 2 is a schematic block diagram of an IMD 100 according to an embodiment. The IMD 100 comprises a sensor connector 110 having connector terminals 111-116 configured to be connected to matching electrode terminals of a sensor arrangement 60 and optionally any implantable medical lead.

In FIG. 2, the sensor connector 110 has been adapted to the particular lead configuration illustrated in FIG. 1. Hence, the sensor connector 110 comprises, in this example, connector terminals 113, 114 configured to be electrically connected to the tip electrode 22 and ring electrode 24 of the RV lead 20 illustrated in FIG. 1. Correspondingly, the connector terminal 115 is configured to be electrically connected to the electrode 32 of the RA lead 30 in FIG. 1.

The sensor connector 110 could also comprise one or more connector terminals 116 configured to be connected to one or more respective case electrodes, which are attached to or forming part of the housing of the IMD 100.

The sensor connector 110 comprises at least one connector terminal 111, 112 configured to be connected to the sensor arrangement 60 comprising at least the first sensor unit and preferably also the second sensor unit. The first sensor unit and the second sensor unit of the sensor arrangement 60 are configured to be arranged in or in connection with the subject's heart to be able to monitor the inter-movement between a basal region of a heart ventricle and an apex of the ventricle during at least a portion of a systolic phase of a cardiac cycle.

In an embodiment, the first sensor unit is preferably configured to be positioned at or in vicinity of the basal region of the ventricle. This basal region generally corresponds to the valve plane, i.e. the plane that separates the upper atria from the lower ventricles. If the IMD 100 is connectable to an atrial lead, such as an RA lead, the sensor unit can advantageously be attached to or form part of the RA lead. The sensor unit is then arranged on the RA lead to be close to the basal region of the right ventricle, i.e. close to the valve plane that separates the right atrium from the right ventricle. The sensor unit is then typically positioned close to the distal end of the RA lead.

In a further alternative the first sensor unit could be positioned in an RV lead. The first sensor unit used for basal monitoring is placed on the RV lead to thereby be positioned close to the base of the right ventricle. Such an approach is also possible for a LV lead that could carry the first sensor unit positioned close to the LV base.

If the sensor arrangement also comprises the second sensor unit, this second sensor unit is provided close to the ventricle apex. For instance, the second sensor unit could be attached to or form part of a distal portion of a ventricular lead. For instance, an RV lead is generally inserted into the right ventricle and attached to the myocardium in connection with the apex of the right ventricle. A sensor unit positioned close to the end of the RV lead will then be positioned in vicinity of the RV apex and also the apex of the heart. Correspondingly, a LV lead is generally inserted into the coronary sinus system of the heart. The distal part of the LV lead will then be positioned close to the apex of the heart and also of the left ventricle. Hence, a sensor unit could be positioned close to the distal end of an LV lead.

In the above mentioned examples, one of the sensor units have been attached to or forms part of a ventricular lead. In another embodiment, the second sensor unit could be provided on a separate ventricular catheter that positions the second sensor unit at or at least close to a ventricular apex. The ventricular catheter does then not need to have any pacing or sensing electrodes and could, for instance, be used solely to correctly position the second sensor unit close to the ventricle apex. Correspondingly, a ventricular catheter or an atrial catheter not having any pacing or sensing electrodes could be used solely for correctly positioning the first sensor unit close to the basal region of a ventricle.

In another embodiment, two sensor-unit-carrying catheters are used with a first (atrial or ventricular) catheter positioning the first sensor unit in proximity to a ventricular basal region and a second (ventricular) catheter positioning the second sensor unit in proximity to the ventricular apex. It is in fact possible to have a single ventricular catheter carrying both the first sensor unit and the second unit. In such a case, an intermediate portion of the ventricular catheter between the first sensor unit (close to the ventricular basal region) and the second sensor unit (close to the ventricular apex) is flexible and elongable, i.e. capable of being elongated.

Many cardiac patients are diagnosed to have an IMD 100 connected to an RA lead and an RV lead. Hence, it is particularly preferred to then position one of the sensor units of the sensor arrangement 60 on the RV lead and the other sensor unit on the RA lead as mentioned in the foregoing.

The sensor arrangement 60 and its at least one sensor unit are configured to generate and output at least one sensor signal representing inter-movement between the basal ventricular region and the ventricle apex. The at least one sensor signal, thus, reflects how the basal ventricular region and the ventricle apex moves relative each other during at least a portion of a systolic phase of a cardiac cycle.

The sensor arrangement 60 could output a single sensor signal or one such sensor signal from each sensor unit, which is further exemplified herein.

In an embodiment, the sensor arrangement 60 comprises only the first sensor unit and outputs a sensor signal representing the inter-movement between the basal ventricular region and the ventricle apex. It is generally sufficient to only monitor the movement of the basal ventricular region during the initial portion of the systolic phase when any elongation of the ventricles take place. The reason for this is that this initial elongation is mainly due to an upward movement of the basal ventricular region whereas the ventricle apex is substantially stationary during this initial elongation. Hence, a sensor signal representing the initial upward movement of the basal ventricular region will be a good approximation of the inter-movement between the basal ventricular region and the ventricle apex during at least a portion of the systolic phase of a cardiac cycle.

The IMD 100 comprises a parameter processor 130. The parameter processor 130 is configured to calculate a contraction status parameter value based on the at least one sensor signal originating from the sensor arrangement 60. This contraction status parameter value represents an elongation of the ventricle following onset of activation of the ventricle during the cardiac cycle. Thus, the parameter processor 130 determines a contraction status parameter value based on the at least one sensor signal to reflect any elongation of the ventricles during an early portion of the systolic phase, i.e. following onset of ventricular activation.

The calculated contraction status parameter value is stored in a memory 140 of the IMD 100 as a diagnostic parameter representing a current contraction status of the heart. Thus, the contraction status parameter value is of diagnostic value and can be used to assess and monitor the contraction status of the subject's heart and detect any deleterious conditions, which might negatively affect the contractility of the heart as previously discussed herein.

In a general embodiment the IMD 100 is able to calculate the contraction status parameter value based only on the monitoring performed by the sensor arrangement 60 of the movement of the basal ventricular region as recorded by the first sensor unit. Although, the elongation of the ventricles during the initial portion of the systolic phase of the cardiac cycle is due to this movement of the basal ventricular region a more accurate representation of the inter-movement between the basal ventricular region and the ventricle apex and thereby a more accurate representation of the elongation of the ventricles is generally obtained by monitoring the movement of not only the basal ventricular region but also of the ventricle apex.

In a particular embodiment the sensor arrangement 60 comprises the first sensor unit and the second sensor unit. The IMD 100 then preferably comprises a distance processor 120 connected to the sensor connector 110 possibly through an optional electronic configuration switch 194. The distance processor 120 thereby receives the at least one sensor signal from the sensor arrangement 60 through the sensor connector 110. The distance processor 120 processes the at least one sensor signal to determine a distance signal representing a distance between the ventricle apex and the basal ventricular region during the at least a portion of the systolic phase.

The particular processing that the distance processor 120 performs based on the at least one sensor signal depends on the type of sensor units of the sensor arrangement 60 and the type of sensor signal. For instance, in an embodiment the sensor signal itself represents the distance between the two sensor units and thereby between the ventricle apex and basal region. In such a case, the distance processor 120 could simply enter the sensor signal as a distance signal in an attached memory 140 or forward the distance signal to a parameter processor 130. In other embodiments, each sensor unit of the sensor arrangement 60 could output a respective sensor signal. The distance processor 120 then determines the distance signal based on these sensor signals, such as by calculating a difference between the sensor signals.

Regardless of the particular processing, the distance processor 120 preferably generates a distance signal having signal samples defining or representing the current distance between the ventricle apex and the basal region during at least a portion of the systolic phase of a cardiac cycle.

In this embodiment the parameter processor 130 is preferably connected to the distance processor 120. The parameter processor 130 is configured to process the distance signal from the distance calculator 120 to calculate the contraction status parameter value. Thus, parameter processor 130 determines the contraction status parameter value based on distance signal samples to reflect any elongation of the ventricles during an early portion of the systolic phase, i.e. following onset of ventricular activation.

As mentioned in the foregoing, the distance signal generated by the distance processor 120 represents the distance between the ventricle apex and basal ventricular region during at least a portion of the systolic phase. In an embodiment, the sensor arrangement 60 could be continuously active to thereby record and forward the at least one sensor signal continuously. In such a case, the distance processor 120 could process the at least one sensor signal to get the distance signal that then represents the apex-basal distance during multiple complete consecutive cardiac cycles. However, such an approach generally drains power quickly from the IMD 100 and its battery 192. In a preferred approach, the sensor arrangement 60 is controlled by a controller 150 to perform the sensor recordings at selected time intervals, such as periodically or upon certain trigger events. These trigger events can be predefined time instances, such as once every week, once every month, etc. A further variant of trigger event is the reception of a trigger message from a non-implantable data processing device, see FIG. 1. The IMD 100 then comprises a receiver or transceiver (TX/RX) 190 with connected antenna 195 to receive such a trigger message. A similar control is also possible without any distance processor 120, wherein the controller 150 instead directly controls the parameter processor 130 to perform the calculation of the contraction status parameter value at selected time intervals.

In these cases the controller 150 controls the sensor arrangement 60 to perform the recordings during a set time interval, such as during 5-10 consecutive cardiac cycles or 10-20 s. The optional distance processor 120 is controlled to generate the distance signal based on the recorded sensor signal(s).

In the above mentioned embodiments, the sensor arrangement 60 could perform the sensor readings during one or multiple complete cardiac cycles. However, the relevant elongation of the ventricles occurs at an early part of the systolic phase of a cardiac cycle. Hence, it is generally sufficient if the sensor arrangement 60 records the at least one sensor signal during at least this early part of the systolic phase during one or multiple cardiac cycles. The relevant early part of the systolic phase is generally from the onset of ventricular activation up to typically no more than half of the systolic phase. Generally, the detection window that captures the relevant early part of the systolic phase could be about 200 ms or shorter and start at the onset of ventricular activation.

Onset of ventricular activation represents the point in time of applying a pacing or stimulation pulse to the ventricle in the case of a paced cardiac cycle or the point in time of a sensed depolarization pulse in the ventricle in the case of an intrinsic cardiac cycle. In general, both these events could be detected by an intracardiac electrogram (IEGM) processor 155 of the IMD 100. The IEGM processor 155 is connected to the sensor connector 110, optionally through the electronic configuration switch 194, and is configured to generate an IEGM signal based on electrical activity of the heart sensed by at least one sensing (and pacing) electrode connected to the sensor connector 110. The onset of ventricular activation could then be defined as the point in time of a QRS complex in the IEGM signal or the point in time of a particular feature in the QRS complex, such as the steepest positive flank on the QRS complex.

If the at least one sensor signal is recorded by the sensor arrangement 60 over multiple cardiac cycles, such as multiple consecutive cardiac cycles, the distance processor 120 could also determine the distance signal to represent the distance between the ventricle apex and basal region for multiple (consecutive) cardiac cycles. In an alternative embodiment, the distance processor 120 determines the distance signal as an average distance signal. Thus, the distance processor 120 could time align the sensor signal from the different cardiac cycles and then calculate the distance signal as an average of the sensor signal(s) over the multiple cardiac cycles. Noise and temporary effects that do not originate from any contraction changes can thereby be repressed by having a distance signal that is determined based on an average of the sensor signal over multiple cardiac cycles.

The parameter processor 130 could also operate to calculate the contraction status parameter value to represent an average elongation of the ventricle for multiple cardiac cycles. In an embodiment, the parameter processor 130 calculates a respective contraction status parameter value for each cardiac cycle of the distance signal or directly based on the at least one sensor signal. The average value of these multiple contraction status parameter values is then output by the parameter processor 130 to the memory 140 as the diagnostic parameter representing the current contraction status of the heart. If the distance signal is an average distance signal as discussed in the foregoing, the parameter processor 130 could calculate a single contraction status parameter value since that parameter value will represent an average elongation of multiple cardiac cycles.

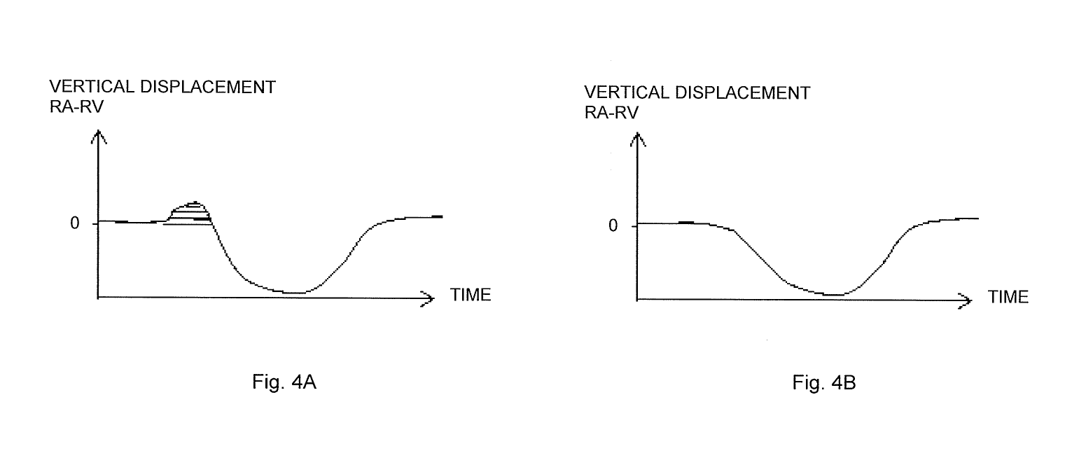

The parameter processor 130 could be configured to calculate the contraction status parameter value according to various embodiments. In an embodiment, the parameter processor 130 identifies the signal sample value that represents the largest elongation of the ventricle, i.e. largest positive sample value, during the relevant early part of the systolic phase in the (average) cardiac cycle. This identified signal sample value is then used as contraction status parameter value. In another embodiment, the parameter processor 130 integrates the detection signal sample values or the sensor signal sample values from the first sensor unit during the relevant early part of the systolic phase. This can be implemented by summing the signal samples that indicate an elongation of the ventricle, i.e. that have a positive signal sample value (if an elongation is indicated with a positive value in the distance signal). FIG. 4A schematically illustrates this approach. The graph represents the distance signal as representing the vertical displacement between a first sensor unit provided in the right atrium (RA) and a second sensor unit provided in the right ventricle (RV), i.e. an embodiment of the distance signal. As is seen in the graph, during the systolic phase the distance between RV apex and basal region initially increases as the ventricle elongates, see hatched region. Thereafter the contraction continues with a shortening of the ventricle, which is seen as negative values in the distance signal.

Summing the signal samples of the distance signal that represents an elongation corresponds to the signal samples of the portion marked with hatching in FIG. 4A. The contraction status parameter value then basically corresponds to the area of the hatched region in FIG. 4A.

The IMD 100 preferably determines a contraction status parameter value at multiple different time instances as mentioned in the foregoing, such as once every week, once every month, etc. The memory 140 will then store these multiple contraction status parameter values. The IMD 100 preferably comprises a transmitter/transceiver 190 that can upload these contraction status parameter values to a non-implantable data processing device, see FIG. 1. There these parameter values can be presented to the subject or, preferably, his/her physician to trend any change in the contractility as assessed based on the multiple contraction status parameter values. For instance, these contraction status parameter values could be plotted over time to visually show any trend in changes in the initial ventricular elongation that would indicate changes or deteriorations in heart contractility.

The recording of the at least one sensor signal by the sensor arrangement 60 is optionally conditioned to occur if certain conditions are met. For instance, the sensor readings could be limited to occur only if the subject's heart rate is within a certain heart rate interval. The reason for such a condition could be that the contraction pattern of a heart could vary slightly depending on the heart rate, in particular the contraction pattern at very high heart rates as compared to the contraction during rest. The memory 140 preferably stores information defining the maximum heart rate and optionally the minimum heart rate at which the IMD 100 can use the sensor signal(s) from the sensor arrangement 60 to calculate the contraction status parameter value. The particular maximum and optional minimum heart rate value can be set by the physician and downloaded to the IMD 100 using the receiver/transceiver 190.

The IMD 100 therefore preferably verifies that the current heart rate is within the allowed heart rate interval before calculating a contraction status parameter value. The current heart rate of the subject can be determined by the controller 150 from the IEGM signal recorded by the IEGM processor 155 according to well-known techniques, i.e. basically determining the time between consecutive R complexes or QRS complexes.

Another condition that can be used by the IMD 100 instead of or as complement to the heart rate condition is patient position. Thus, the contraction pattern of the subject's heart may differ slightly depending on whether the subject is standing or lying down, or whether the subject is lying in a supine position or on the side. The IMD 100 then preferably comprises a position sensor (not illustrated) arranged inside the housing of the IMD 100 or outside of the housing and connected to the IMD 100 through the sensor connector 110. The position sensor then generates a position signal that represents the current position of the subject. The controller 150 processes this position signal to verify that the subject has a target position, such as standing up or lying in a supine position, prior to calculating the contraction status parameter value.

The above disclosed embodiments that provide a conditioned calculation of the contraction status parameter value are particularly suitable when the IMD 100 is configured to calculate the contraction status parameter value at different time instances, such as once per week, once per month or more seldom. The condition(s) imposed by the IMD 100 lead(s) to that the contraction status parameter values can be compared to each other and be used to detect any sudden changes or trends in contraction status over time.

The memory 140 of the IMD 100 in FIG. 2 preferably not only stores the contraction status parameter value calculated by the parameter processor 130. The memory 140 advantageously also stores a reference parameter value representing a reference elongation of the ventricle. This reference parameter value could be a previously calculated contraction status parameter value obtained from the parameter processor 130. In such a case, this previous contraction status parameter value is calculated by the parameter processor 130 during a period of time when it is concluded that the subject is not suffering from any immediate heart condition that impairs the contraction status of his/her heart. This can be verified in connection with a visit to the subject's physician. The reference parameter value could also be an average of previously calculated contraction status parameter values.

Alternatively, the reference parameter value could be set by the physician to represent an average elongation of the ventricle specified for an average human heart. The set reference parameter value is then downloaded to the IMD 100 and the memory 140.

The IMD 100 preferably comprises a status processor 160 configured to compare the contraction status parameter value calculated by the parameter processor 130 with the reference parameter value. The status processor 160 further generates a contraction status notification if the elongation of the ventricle as represented by the contraction status parameter value is significantly shorter than a reference elongation of the ventricle as represented by the reference parameter value. Thus, if the current elongation is smaller than the reference elongation, or preferably differs from, i.e. is smaller than, the reference elongation with more than a defined delta value, the status processor 160 generates the contraction status notification. The contraction status notification is therefore generated if the elongation of the ventricle is absent or is reducing, which indicate an impaired contraction status of the heart.

FIGS. 4A and 4B illustrate this concept. In FIG. 4A, the contraction status of the heart is fine with a distinct elongation of the right ventricle during the early part of the systolic phase. In FIG. 4B this elongation is gone. Hence the contractility of the heart has become impaired due to some deleterious condition, such as ischemia.

The contraction status notification, if generated, is then preferably, at least temporarily, entered in the memory 140. The contraction status notification can be uploaded to the non-implantable data processing device (see FIG. 1) by the transmitter/transceiver 190 of the IMD 100. This uploading can be performed automatically when the IMD 100 is within communication distance to the data processing device or upon an explicit interrogation from the data processing device. The contraction status notification will then inform the subject or his/her physician that the ventricular elongation has reduced to be less than the reference elongation and that this could be an indication of impaired contraction status.

The contraction status notification could also or alternatively be used by the controller 150 to apply pacing pulses according to a pacing scheme that is selected to combat any deterioration in contractility as determined from the contraction status notification. The IMD 100 then preferably comprises a ventricular pulse generator 170 configured to generate pacing pulses to be applied to a ventricle of the heart using electrode(s) of a connected ventricular lead. The IMD 100 may in addition or alternatively comprise an atrial pulse generator 175 configured to generate pacing pulses to be applied to an atrium of the heart using electrode(s) of a connected atrial lead. The controller 150 is configured to control the ventricular and atrial pulse generators 170, 175 to generate and apply pacing pulses according to a pacing scheme defined by the controller 150. In an embodiment, the controller 150 has access to at least two different such pacing schemes: a default pacing scheme and a contraction improving pacing scheme. The default pacing scheme is the pacing scheme normally used by the controller 150 and the IMD 100. However, if the status processor 160 generates the contraction status notification that indicates that the current contraction status is impaired due to absence of or a reduced ventricular elongation, the controller 150 could be configured to switch from the default pacing scheme to the contraction improving pacing scheme. This contraction improving pacing scheme is selected by the physician to strengthen and improve the contractility of the heart to thereby combat and compensate for any cardiac condition, such as ischemia, that can result in a (temporary) deterioration of the contractility of the heart. The contraction improving pacing scheme could, for instance, use a different atrioventricular delay (AVD) and/or a different interventricular delay (VVD) as compared to the default pacing scheme.

The sensor arrangement 60 that is connectable to the IMD 100 preferably comprises a first sensor configured to output a basal sensor signal representing movement of the basal ventricular region during at least a portion of the systolic phase. This first sensor is then arranged as previously discussed herein in connection with the basal region of the ventricle and with a second sensor arranged at or close to the ventricle apex. The second sensor is then configured to output an apical sensor signal representing movement of the apex during at least a portion of the systolic phase.

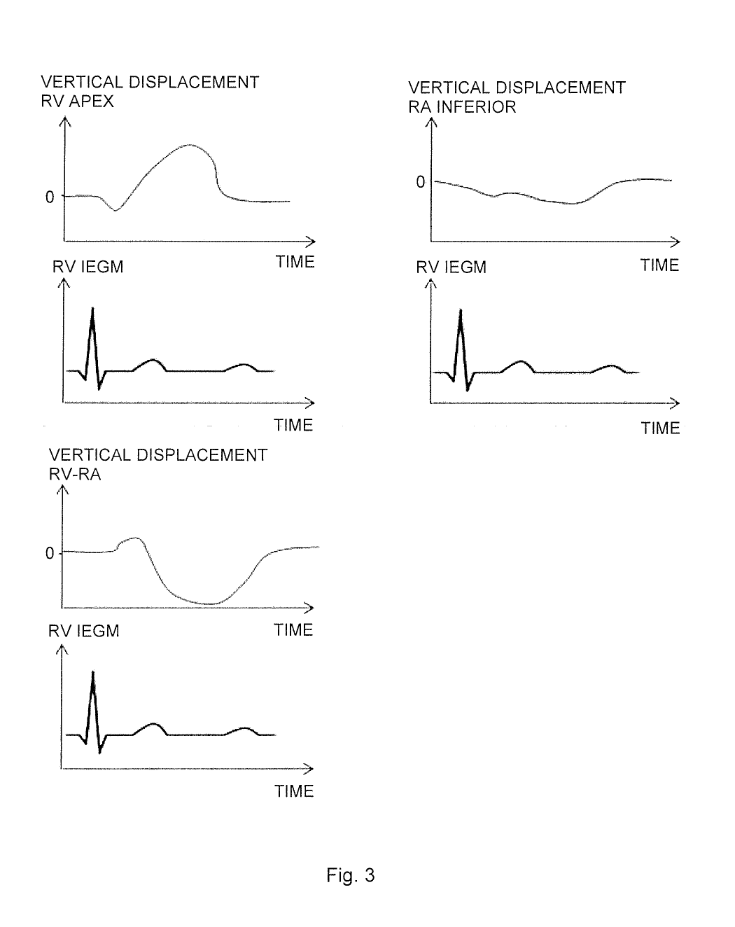

FIG. 3 schematically illustrates the sensor signals from these two sensors during a systolic phase of a cardiac cycle. The upper left diagram illustrates the movement of the RV apex and represents the apical sensor signal. The diagram also shows an IEGM signal recorded using a RV electrode during the cardiac cycle. The upper right diagram illustrates the corresponding movement of the valve plane, i.e. the basal region of the right ventricle, and represents the basal sensor signal. The distance processor 120 could then be configured to determine the distance signal based on a difference between the apical sensor signal and the basal sensor signal, as illustrated in the lower right diagram. In this embodiment, the distance processor 120 uses the IEGM signal recorded together with the apical and basal sensor signals to align the two sensor signals with each other with regard to time, typically by identifying the respective sensor samples that coincide with a defined feature in the IEGM signal, such as maximum or minimum in QRS complex or steepest positive flank of QRS complex. The distance signal is then preferably calculated by subtracting the samples of one of the apical and basal signals from the corresponding time-aligned samples of the other of the apical and basal signals as shown in the lower left diagram of FIG. 3. In this case both sensors have the same sampling rate. If the sampling rate is different the distance processor 120 has to compensate for this sampling-difference prior to performing the sample subtraction.

In a particular embodiment, the first sensor is a first accelerometer arranged an RA lead. The second sensor is then a second accelerometer arranged in connection with a distal end of a RV lead. Another embodiment uses a first position sensor on the RA lead with a second position sensor on the RV lead.

A further variant is to use an ultrasound emitter and an ultrasound receiver as the first and second sensor units of the sensor arrangement 60. The RV lead then comprises one of the ultrasound emitter and the ultrasound receiver with the other one arranged on the RA lead. The ultrasound emitter emits an ultrasound signal that is captured by the ultrasound receiver. The intensity of the captured ultrasound signal is correlated to the distance between the ultrasound emitter and receiver and can therefore be used as sensor signal of the sensor arrangement 60. Alternatively, the ultrasound receiver could be configured to measure the time from transmission of the ultrasound signal at the ultrasound transmitter until the ultrasound signal is received by the ultrasound receiver. The recorded time periods could then be used as sensor signal.

The IMD 100 of FIG. 2 may optionally also comprise circuits for sensing electrical activity of the heart. Such circuits can be in the form of a ventricular sensing circuit 180 and/or an atrial sensing circuit 185. The ventricular and atrial sensing circuits 180, 185 of the IMD 100 may include dedicated sense amplifiers, multiplexed amplifiers, or shared amplifiers. The electronic configuration switch 194 determines the “sensing polarity” of the cardiac signal by selectively closing the appropriate switches, as is also known in the art. In this way, the clinician may program the sensing polarity independent of the stimulation polarity. The sensing circuits are optionally capable of obtaining information indicative of tissue capture.

Each sensing circuit 180, 185 preferably employs one or more low power, precision amplifiers with programmable gain and/or automatic gain control, band-pass filtering, and a threshold detection circuit, as known in the art, to selectively sense the cardiac signal of interest.

The outputs of the ventricular and atrial sensing circuits 180, 185 are connected to the controller 150, which, in turn, is able to trigger or inhibit the ventricular and atrial pulse generators 170, 175, respectively, in a demand fashion in response to the absence or presence of cardiac activity in the appropriate chambers of the heart.

The controller 150 of the IMD 100 is preferably in the form of a programmable microcontroller 150 that controls the operation of the IMD 100. The controller 150 typically includes a microprocessor, or equivalent control circuitry, designed specifically for controlling the delivery of pacing therapy, and may further include RAM or ROM memory, logic and timing circuitry, state machine circuitry, and I/O circuitry. Typically, the controller 150 is configured to process or monitor input signals as controlled by a program code stored in a designated memory block. The type of controller 150 is not critical to the described implementations. In clear contrast, any suitable controller may be used that carries out the functions described herein. The use of microprocessor-based control circuits for performing timing and data analysis functions are well known in the art.

Furthermore, the controller 150 is also typically capable of analyzing information output from the sensing circuits 180, 185 to determine or detect whether and to what degree tissue capture has occurred and to program a pulse, or pulse sequence, in response to such determinations. The sensing circuits 180, 185, in turn, receive control signals over signal lines from the controller 150 for purposes of controlling the gain, threshold, polarization charge removal circuitry, and the timing of any blocking circuitry coupled to the inputs of the sensing circuits 180, 185 as is known in the art.

The optional electronic configuration switch 194 includes a plurality of switches (not shown) for connecting the desired connector terminals 111-116 to the appropriate I/O circuits, thereby providing complete electrode programmability. Accordingly, the electronic configuration switch 194, in response to a control signal from the controller 150, determines the polarity of the stimulating pulses by selectively closing the appropriate combination of switches as is known in the art.

While a particular multi-chamber device is shown in FIG. 2, it is to be appreciated and understood that this is done merely for illustrative purposes. Thus, the techniques and methods described below can be implemented in connection with other suitably configured IMDs. Accordingly, the person skilled in the art can readily duplicate, eliminate, or disable the appropriate circuitry in any desired combination.

The IMD 100 additionally includes a battery 180 that provides operating power to all of the circuits shown in FIG. 2.

In FIG. 2, the optional distance processor 120, the parameter processor 130 and the optional status processor 160 have been illustrated as being run by the controller 150. These units 120, 130, 160 can then be implemented as a computer program product stored in the memory 140 and loaded and run on a general purpose or specially adapted computer, processor or microprocessor, represented by the controller 150 in FIG. 2. The software includes computer program code elements or software code portions effectuating the operation of the units 120, 130, 160. The program may be stored in whole or part, on or in one or more suitable computer readable media or data storage means that can be provided in an IMD 100.

In an alternative approach, the units 120, 130, 160 are implemented as hardware circuits in the IMD 100, preferably connected to the controller 150, such as in the form of special purpose circuits, such as ASICs (Application Specific Integrated Circuits).

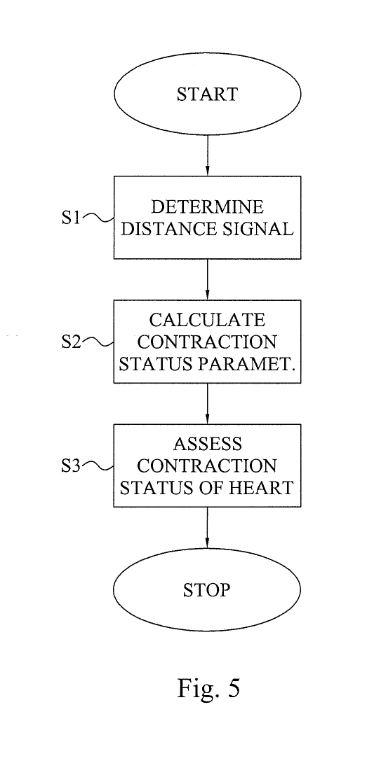

FIG. 5 is flow diagram illustrating a method of assessing contraction status of a heart 15 in a subject. The method comprises determining a distance signal in step S1, where the distance signal represents a distance between an apex of a heart ventricle and a basal region of the ventricle during at least a portion of a systolic phase of a cardiac cycle. A next step S2 calculates, based on the distance signal, a contraction status parameter value representing an elongation of the ventricle following onset of activation of the ventricle during the cardiac cycle. The contraction status parameter value calculated in step S2 is used in step S3 to assess the contraction status of the heart.

The assessment performed in step S3 could be performed by comparing the contraction status parameter value with a reference parameter value, such as a predefined threshold value or a previously determined contraction status parameter value as previously discussed herein.

The method of steps S1 to S3 is preferably performed at different times to thereby monitor and trend contraction status over time.

The method of FIG. 5 can be performed using an IMD as previously disclosed herein. In an alternative embodiment, the method could be performed by a contraction status assessing system in a catheterization laboratory (cath lab), such as in connection with implanting an IMD. FIG. 6 schematically illustrates such an approach. This embodiment uses a catheter or stylet 50 comprising a sensor arrangement comprising a sensor 52 configured to output an apical sensor signal representing movement of the ventricular apex when the sensor is position in connection with the ventricular apex. The catheter/stylet 50 is then moved to position the sensor 52 in connection with the basal ventricular region to thereby output a reference sensor signal representing movement of the basal region of the ventricle. The opposite procedure is of course possible with basal measurements prior to apical measurements.

This procedure can be conducted using a so-called MediGuide sensor coil 52 as sensor arranged on the catheter/stylet 50. In such a case, once the sensor coil 52 is in position, such as in connection with the RV apex, about 10-20 s of the medical position system (MPS) signal (also sometimes referred to as medical global positioning system (medical GPS) signal) is recorded and also an RV IEGM signal. Then the catheter/stylet 50 is moved either for RA lead implantation or simply positions the sensor coil 52 in the lower part of the inter-atrial septum or adjacent to the tricuspid valve. Once more about 10-20 s of the MPS signal is recorded as well as the RV IEGM signal.

The recorded data is stored in a data processing device 200, such as a programmer or pacemaker system analyzer (PSA) connected to the sensor coil 52. The data processing device 200 synchronizes the two data segments, i.e. the MPS signal from the apex and from the valve plane, using the RV IEGM signal. This is easily done by optionally applying filtering, such as standard pacemaker IEGM filters, and, for instance, locating the steepest positive flank on the QRS complex or some other predefined IEGM feature. The two data sets are then aligned based on the identified IEGM features. Before or after the time alignment, the two data sets could be averaged over time to eliminate high-frequency noise and potential respiratory components.

The two (averaged and aligned) data sets corresponding to the apical sensor signal and the reference sensor signal are then used to determine the distance signal, such as a difference between the two data sets.

During the main part of the systole the distance signal will diminish but in a healthy heart there is an initial elongation of the ventricles causing in fact a temporary small increase in the distance signal. The data processing device 200 therefore calculates the contraction status parameter value based on the distance signal by analyzing the first part of the distance signal immediately following the detected QRS and looking for any positive components.

One implementation to calculate the contraction status parameter value is to integrate, in practice sum up, all positive samples in the distance signal during a window of, for instance, about 200 ms following the detected QRS complex, to generate a scalar output that could either be Boolean variable (contraction status notification) to simply state if the elongation is present or nor, or a decimal number to be used as more high resolution diagnostic parameter (contraction status parameter value).

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

I claim as my invention:

1. An implantable medical device comprising:
a sensor connector connectable to a sensor arrangement comprising at least a first sensor unit, said sensor arrangement is configured to output, to said sensor connector, at least one sensor signal representing inter-movement between a basal region of a ventricle in a heart of a subject and an apex of said ventricle during at least a portion of a systolic phase of a cardiac cycle;
a parameter processor configured to calculate, based on said at least one sensor signal, a contraction status parameter value representing an elongation of said ventricle following onset of activation of said ventricle during said cardiac cycle; and
a memory configured to store said contraction status parameter value as a diagnostic parameter representing a current contraction status of said heart.

2. The implantable medical device according to claim 1, wherein said sensor connector is connectable to said sensor arrangement comprising said first sensor unit configured to be arranged in connection with said basal region of said ventricle.

3. The implantable medical device according to claim 1, wherein said sensor connector is connectable to said sensor arrangement comprising said first sensor unit and a second sensor unit, said implantable medical device further comprising a distance processor configured to process said at least one sensor signal received by said sensor connector to determine a distance signal representing a distance between said apex and said basal region during said at least a portion of said systolic phase, wherein said parameter processor is connected to said distance processor and configured to process said distance signal to calculate said contraction status parameter value representing said elongation of said ventricle following onset of activation of said ventricle during said cardiac cycle.

4. The implantable medical device according to claim 3, wherein said sensor connector is connectable to said sensor arrangement comprising said first sensor configured to output a basal sensor signal representing movement of said basal region during said at least a portion of said systolic phase and said second sensor configured to output an apical sensor signal representing movement of said apex during said at least a portion of said systolic phase.

5. The implantable medical device according to claim 4, wherein said sensor connector is connectable to a right atrial lead comprising a first accelerometer and a right ventricular lead comprising a second accelerometer arranged in connection with a distal end of said right ventricular lead.

7. The implantable medical device according to claim 4, wherein said sensor connector is connectable to a right atrial lead comprising a first position sensor and a right ventricular lead comprising a second position sensor arranged in connection with a distal end of said right ventricular lead.

8. The implantable medical device according to claim 4, wherein said distance processor is configured to determine said distance signal based on a difference between said basal sensor signal and said apical sensor signal.

9. The implantable medical device according to claim 3, wherein said sensor connector is connectable to a right atrial lead comprising one of an ultrasound emitter and an ultrasound receiver and a right ventricular lead comprising the other of said ultrasound emitter and said ultrasound receiver arranged in connection with a distal end of said right ventricular lead, said sensor connector is configured to receive a sensor signal from said ultrasound receiver representing inter-movement between said basal region and said apex during said at least a portion of said systolic phase.

10. The implantable medical device according to claim 3, wherein said parameter processor is configured to calculate said contraction status parameter value by summing the signal samples of said distance signal that indicate an elongation of said ventricle during said at least a portion of said systolic phase.

11. The implantable medical device according to claim 1, wherein said memory comprises a reference parameter value representing a reference elongation of said ventricle and said implantable medical device comprises a status processor configured to compare said contraction status parameter value with said reference parameter value and generate a contraction status notification if said elongation of said ventricle as represented by said contraction status parameter value is shorter than said reference elongation of said ventricle as represented by said reference parameter value, wherein said memory is configured to store said contraction status notification.

12. The implantable medical device according to claim 1, further comprising:
an intracardiac electrogram, IEGM, processor configured to generate an IEGM signal based on electric activity of said heart sensed by at least one electrode connectable to the sensor connector; and
a controller connected to said IEGM processor and said distance processor and configured to i) determine a current heart rate of said heart based on said IEGM signal and ii) control said parameter processor to calculate said contraction status parameter value if said current heart rate is within a defined heart rate interval.

13. The implantable medical device according to claim 1, wherein said implantable medical device comprises or is connectable to a position sensor configured to generate a position signal representing a current position of said subject, said implantable medical device comprises a controller connected to said parameter processor to calculate said contraction status parameter value if said current position is equal to a target position as determined based on said position signal.

14. A method of assessing contraction status of a heart in a subject comprising:
determining a distance signal representing a distance between an apex of a ventricle in said heart and a basal region of said ventricle during at least a portion of a systolic phase of a cardiac cycle;
calculating, based on said distance signal, a contraction status parameter value representing an elongation of said ventricle following onset of activation of said ventricle during said cardiac cycle; and
assessing said contraction status of said heart based on said contraction status parameter value.
